(19) United States
(12) Patent Application Publication
(10) Pub. No.: US 2017/0151143 A1
(43) Pub. Date: Jun. 1, 2017

(54) HIGH-PERFORMANCE HAIR TREATMENT AGENTS HAVING ANTI-WASHOUT EFFECT

(71) Applicant: Henkel AG & Co. KGaA, Duesseldorf (DE)

(72) Inventors: Volker Scheunemann, Lueneburg (DE); Erik Schulze zur Wiesche, Hamburg (DE)

(73) Assignee: Henkel AG & Co. KGaA, Duesseldorf (DE)

(21) Appl. No.: 15/358,918

(22) Filed: Nov. 22, 2016

(30) Foreign Application Priority Data
Dec. 1, 2015 (DE) 10 2015 223 832.6

(51) Int. Cl. A61K 8/43; A61K 8/46; A61K 8/42; A61Q 17/00; A61K 8/362; A61K 8/67; A61Q 5/00; A61Q 5/12; A61Q 5/02; A61K 8/23

(52) U.S. Cl. CPC A61K 8/43; A61Q 5/02; A61K 8/463; A61K 8/42; A61K 8/23; A61K 8/362; A61K 8/675; A61Q 5/004; A61Q 5/12; A61Q 17/005; A61K 2800/596

(57) ABSTRACT

Hair treatment agents, comprising at least one anionic surfactant, at least one amphoteric surfactant, at least one divalent or trivalent metal salts, at least one organic acid and ethyl lauroyl arginate reduce or prevent dye from being washed out of colored hair.

### CROSS-REFERENCE TO RELATED APPLICATION

This application claims priority to German Patent Application No. 10 2015 223 832.6, filed Dec. 1, 2015, which is incorporated herein by reference in its entirety.

### TECHNICAL FIELD

This disclosure relates to hair treatment agents, in particular to shampoos and so-called conditioners, comprising an active ingredient combination for gentle and effective hair care.

### BACKGROUND

Due in no small part to severe stressing of the hair, for example as a result of coloring or permanent-wave treatment, but also as a result of cleaning the hair with shampoos and due to environmental pollution, the significance of care products having a preferably long-lasting effect is on the rise. Such care agents influence the natural structure and properties of the hair. Subsequent to such care treatments, for example, the wet and dry combability of the hair, and the hold and volume of the hair may be optimized, or the hair may be protected from increased split ends.

It has therefore been customary for some time to apply a special after-treatment to hair. To this end, the hair is treated with special active ingredients, usually in the form of a rinse conditioner, such as quaternary ammonium salts or special polymers. Depending on the formulation, this treatment improves the combability, hold and volume of the hair and reduces split ends.

Likewise, multifunctional cosmetic products are known in the prior art. These include in particular what are known as “2-in-1” shampoos, which not only clean, but also condition the hair. These products are valued highly by the consumer since the product performance thereof eliminates at least one method step, for example conditioning using a traditional hair rinse conditioner.

In addition, products used to change the natural hair color play a prominent role in hair cosmetics. A distinction is made between permanent, semi-permanent and temporary coloring systems, which are based on chemical and/or natural dyes. Hair colors artificially created by permanent, semi-permanent or temporary coloring systems, however, have the disadvantage that these may undesirably change, for example during or after cleaning the hair.

An “undesirable change” shall be understood to mean fading or bleeding and the loss of luster of the hue of the hair achieved by the particular coloring process. These changes may be further amplified by environmental factors and/or sunlight.

The use of divalent metal salts in hair coloring agents to improve the longevity, and thus the fastness, of the coloration is known from EP 2438900 A1.

A continued need exists for active ingredients or active ingredient combinations for hair treatment agents having good nourishing properties, which additionally strengthen the adherence of dyes to the hair fibers and thereby preserve the fastness of the artificially created hair color, and for further development of hair treatment agents in this regard.

However, it has been observed that the hardness of the water can at times adversely influence the nourishing properties of hair treatment agents, so that the care performance of one and the same product may be either too high or too low, depending on the quality of the water. Excessively high care performance or “overcare” (development of a so-called build-up effect) of the hair shall be understood to mean in particular a greasy feel of the hair, low volume of the hair and/or a stringy, unkempt appearance of the hair, while hair that is damaged (brittle, susceptible to split ends, dull) is an indication of insufficient care performance.

### BRIEF SUMMARY

Hair treatment agents and methods for treating hair using the hair treatment agents are provided. In accordance with an exemplary embodiment, a hair treatment agent comprises at least one anionic surfactant; at least one amphoteric surfactant; at least one divalent or trivalent metal salt; at least one organic acid; and ethyl lauroyl arginate.

In accordance with another exemplary embodiment, a method for treating hair comprises the steps of applying to dry or moist hair a hair treatment agent comprising an anionic surfactant; an amphoteric surfactant; a divalent or trivalent metal salt; an organic acid; and ethyl lauroyl arginate. The hair treatment agent is rinsed from the hair after the hair treatment agent is left on the hair for a period of from about 30 to about 300 seconds.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

This detailed description provides easily skin-tolerable and nourishing hair treatment agents that have outstanding foam properties and are easy to rinse off using water, and that moreover reduce or prevent dye from being washed out of colored hair.

The nourishing hair treatment agents offer a consistent conditioning performance, regardless of the water quality, and in particular do not weigh down fine hair and/or damaged hair, while strengthening the structure of the hair, thus protecting it from split ends and breakage, and improving combability and the feel. Moreover, the agents act in a bacteria-reducing manner.

It has now been found that a combination of certain ingredients has a particularly positive effect on colored hair treated therewith and the hair follicles.

A first embodiment of the present invention is hair treatment agents, comprising:

a) at least one anionic surfactant;

b) at least one amphoteric surfactant;

c) at least one divalent or trivalent metal salt.

d) at least one organic acid;

e) Ethyl lauroyl arginate.

Hair treatment agents as contemplated herein are, for example, hair shampoos, hair conditioners, conditioning shampoos, hair sprays, hair rinse conditioners, deep hair conditioners, hair packs, hair tonics, permanent wave fixation solutions, hair coloring shampoos, hair coloring agents, hair setting lotions, hair style setting agents, hair styling preparations, blow drying lotions, mousse, hair gels, hair waxes or the combinations thereof. With respect to the fact that men in particular often shy away from the use of several different products and/or multiple application steps, agents that men use anyhow are preferred. Preferred agents are therefore shampoos, conditioning agents or hair tonics.

The hair treatment agents comprise at least one anionic surfactant. All anionic surface-active substances that are suitable for use on the human body are suitable anionic surfactants and emulsifiers for the compositions contemplated herein. These are characterized by a water-soluble-rendering anionic group, such as a carboxylate, sulfate, sulfonate or phosphate group and a lipophilic alkyl group having approximately 8 to 30 carbon atoms. In addition, glycol or polyglycol ether groups, ester, ether and amide groups and hydroxyl groups can be present in the molecule. Examples of suitable anionic surfactants and emulsifiers, each in the form of the sodium, potassium and ammonium salts, and monoalkanol, dialkanol and trialkanol ammonium salts having 2 to 4 carbon atoms in the alkanol group, are:
linear and branched fatty acids having 8 to 30 carbon atoms (soaps);
ether carboxylic acids of formula R—O—(CH₂—CH₂O)ₓ—CH₂—COOH, in which R is a linear alkyl group having 8 to 30 carbon atoms, and x=0 or 1 to 16;
acyl sarcosides having 8 to 24 carbon atoms in the acyl group;
acyl taurides having 8 to 24 carbon atoms in the acyl group;
acyl isethionates having 8 to 24 carbon atoms in the acyl group;
linear alkane sulfonates having 8 to 24 carbon atoms;
linear alpha-olefin sulfonates having 8 to 24 carbon atoms;
alpha-sulfo fatty acid methyl esters of fatty acids having 8 to 30 carbon atoms;
acyl glutamates of formula (T-I),

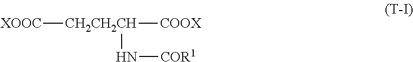

in which R¹CO denotes a linear or branched acyl group having 6 to 22 carbon atoms and 0, 1, 2 or 3 double bonds, and X denotes hydrogen, an alkali metal and/or alkaline earth metal, ammonium, alkyl ammonium, alkanol ammonium or glucammonium, such as acyl glutamates derived from fatty acids having 6 to 22, especially 12 to 18, carbon atoms, such as C_{12/14} or C_{12/18} coconut fatty acid, lauric acid, myristic acid, palmitic acid and/or stearic acid, in particular sodium-N-cocoyl and sodium-N-stearoyl-L-glutamate;
esters of a hydroxy-substituted dicarboxylic or tricarboxylic acid of the general formula (T-II),

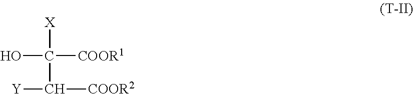

in which X=H or a CH₂COOR group, Y=H or OH, on condition that Y=H when X=—CH₂COOR, R, R¹ and R², independently of one another, denote a hydrogen atom, an alkali metal or alkaline earth metal cation, an ammonium group, the cation of an ammonium-organic base or a group Z, which stems from a polyhydroxylated organic compound selected from the group of the etherified (C₆-C₁₈) alkyl polysaccharides having 1 to 6 monomeric saccharide units and/or the etherified aliphatic (C₆-C₁₆) hydroxyalkyl polyols having 2 to 16 hydroxyl groups, with the proviso that at least one of the groups R, R¹ or R² is a group Z;
esters of sulfosuccinic acid or of sulfosuccinates of the general formula (T-III),

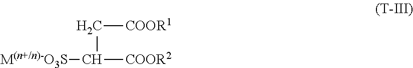

in which M^{(n+/n)} for n=1 represents a hydrogen atom, an alkali metal cation, an ammonium group or the cation of an ammonium-organic base, and for n=2 represents an alkaline earth metal cation, and R¹ and R² independently of one another denote a hydrogen atom, an alkali metal or alkaline earth metal cation, an ammonium group, the cation of an ammonium-organic base or a group Z, which stems from a polyhydroxylated organic compound selected from the group of the etherified (C₆-C₁₈) alkyl polysaccharides having 1 to 6 monomeric saccharide units and/or the etherified aliphatic (C₆-C₁₆) hydroxyalkyl polyols having 2 to 16 hydroxyl groups, with the proviso that at least one of the groups R¹ or R² is a group Z;
sulfosuccinic acid monoalkyl and dialkyl esters having 8 to 24 carbon atoms in the alkyl group, and sulfosuccinic acid monoalkyl polyoxyethyl esters having 8 to 24 carbon atoms in the alkyl group and 1 to 6 oxyethyl groups;
alkyl sulfates and alkyl polyglycol ether sulfates of formula R—(O—CH₂—CH₂)ₓ—OSO₃H, in which R is a preferably linear alkyl group having 8 to 30 carbon atoms, and x=0 or 1 to 12;
mixed surface-active hydroxysulfonates according to DE-A-37 25 030 (U.S. Pat. No. 4,865,774);
esters of tartaric acid and citric acid with alcohols that represent addition products of approximately 2 to 15 molecules ethylene oxide and/or propylene oxide to C₈₋₂₂ fatty alcohols;
alkyl and/or alkenyl ether phosphates;
sulfated fatty acid alkylene glycol esters;
monoglyceride sulfates and monoglyceride ether sulfates.

Preferred anionic surfactants and emulsifiers are acyl glutamates, acyl isethionates, acyl sarcosinates and acyl taurates, each comprising a linear or branched acyl group having 6 to 22 carbon atoms and 0, 1, 2 or 3 double bonds, which in particularly preferred embodiments is selected from an octanoyl, a decanoyl, a lauroyl, a myristoyl, a palmitoyl and a stearoyl group, esters of tartaric acid, citric acid or succinic acid or of the salts of these acids with alkylated glucose, in particular the products by the INCI names Disodium Coco-Glucoside Citrate, Sodium Coco-Glucoside Tartrate and Disodium Coco-Glucoside Sulfosuccinate, alkyl polyglycol ether sulfates and ether carboxylic acids having 8 to 18 carbon atoms in the alkyl group and up to 12 ethoxy groups in the molecule, sulfosuccinic acid monoalkyl and dialkyl esters having 8 to 18 carbon atoms in the alkyl group, and sulfosuccinic acid monoalkyl polyoxyethyl esters having 8 to 18 carbon atoms in the alkyl group and 1 to 6 ethoxy groups.

Further preferred anionic surfactants are alkyl sulfates, alkyl polyglycol ether sulfates and ether carboxylic acids having 10 to 18 carbon atoms in the alkyl group, and up to 12 glycol ether groups in the molecule, sulfosuccinic acid monoalkyl and dialkyl esters having 8 to 18 carbon atoms in the alkyl group, and sulfosuccinic acid monoalkyl polyoxyethyl esters having 8 to 18 carbon atoms in the alkyl group and 1 to 6 oxyethyl groups.

Particularly preferred anionic surfactants are the alkali or ammonium salts of lauryl ether sulfate having a degree of ethoxylation of 2 to 4 EO.

Preferred hair treatment agents comprise, based on the weight thereof, about 0.5 to about 20 wt. %, especially about 0.75 to about 15 wt. %, more preferably about 1 to about 12 wt. %, and in particular about 2 to about 10 wt. % anionic surfactant(s).

In particular, preferred hair treatment agents as contemplated herein are characterized by comprising, based on the weight thereof, about 0.5 to about 20 wt. %, especially about 0.75 to about 15 wt. %, more preferably about 1 to about 12 wt. %, and in particular about 2 to about 10 wt. % alkyl (ether) sulfates of the general formula R—(OCH₂—CH₂)ₙ—OSO₃X, in which R denotes a straight-chain or branched, saturated or unsaturated alkyl group having 8 to 24 carbon atoms, n denotes the number 0 or 1 to 12, and X denotes an alkali, alkaline earth, ammonium or alkanolamine ion.

The hair treatment agents contemplated herein comprise at least one amphoteric surfactant. Amphoteric surfactants, also referred to as zwitterionic surfactants, are considered to be surfactants that include both a negatively charged and a positively charged functional group.

Particularly suited zwitterionic surfactants are those known as betaines, such as N-alkyl-N,N-dimethylammonium glycinates, for example coconut alkyl dimethylammonium glycinate, N-acylaminopropyl-N,N-dimethylammonium glycinate, for example, coconut acylaminopropyl dimethylammonium glycinate, and 2-alkyl-3-carboxymethyl-3-hydroxyethyl imidazolines, each having 8 to 18 carbon atoms in the alkyl or acyl group, and coconut acylamino ethylhydroxyethyl carboxymethyl glycinate. A preferred zwitterionic surfactant is the fatty acid amide derivative known by the INCI name Cocamidopropyl Betaine. Further examples of suitable ampholytic surfactants are N-alkyl glycines, N-alkylaminopropionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids, and alkylaminoacetic acids, each having approximately 8 to 24 carbon atoms in the alkyl group. Particularly preferred ampholytic surfactants are N-cocoalkylaminopropionate, cocoacylaminoethyl aminopropionate and C₁₂ to C₁₈ acyl sarcosine.

Preferred hair treatment agents contemplated herein comprise, based on the weight thereof, about 0.3 to about 10 wt. %, especially about 0.5 to about 8 wt. %, more preferably about 0.75 to about 6 wt. %, and in particular about 1 to about 5 wt. % amphoteric surfactant(s).

Particularly preferred hair treatment agents contemplated herein are characterized by comprising amphoteric surfactant(s) from the following groups:
N-alkyl glycines;
N-alkyl propionic acids;
N-alkylaminobutyric acids;
N-alkyliminodipropionic acids;
N-hydroxyethyl-N-alkylamidopropyl glycines;
N-alkyl taurines;
N-alkyl sarcosines;
2-alkylaminopropionic acids, each having approximately 8 to 24 carbon atoms in the alkyl group;
alkylaminoacetic acids, each having approximately 8 to 24 carbon atoms in the alkyl group;
N-cocoalkylaminopropionate;
cocoacylaminoethyl aminopropionate;
C₁₂ to C₁₈ acyl sarcosine;
N-alkyl-N,N-dimethylammonium glycinates, for example coconut alkyl dimethylammonium glycinate;
N-acylaminopropyl-N,N-dimethylammonium glycinate, for example, coconut acylaminopropyl dimethylammonium glycinate;
2-alkyl-3-carboxymethyl-3-hydroxyethyl imidazolines, each having 8 to 18 carbon atoms in the alkyl or acyl group;
coconut acylamino ethylhydroxyethyl carboxymethyl glycinate;
the compounds known by the INCI name Cocamidopropyl Betaine;
the compounds known by the INCI name Disodium Cocoamphodiacetate,
   wherein preferred agents comprise the amphoteric surfactant or surfactants in amounts of about 0.3 to about 10 wt. %, especially about 0.5 to about 8 wt. %, more preferably about 0.75 to about 6 wt. %, and in particular about 1 to about 5 wt. %, in each case based on the total agent.

Particularly preferred hair treatment agents comprise betaines of formula (Bet-I) as amphoteric surfactants

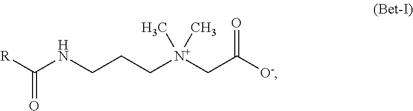

in which R denotes a straight-chain or branched, saturated, monounsaturated or polyunsaturated alkyl or alkenyl group having 8 to 24 carbon atoms.

According to INCI nomenclature, these surfactants are referred to as Amidopropylbetaines, wherein representatives derived from coconut fatty acids are preferred and referred to as Cocoamidopropylbetaines. As contemplated herein, particularly preferably surfactants of formula (Bet-I) that are a mixture of the following representatives are used:

H₃C—(CH₂)₇—C(O)—NH—(CH₂)₃N⁺(CH₃)₂CH₂COO⁻

H₃C—(CH₂)₉—C(O)—NH—(CH₂)₃N⁺(CH₃)₂CH₂COO⁻

H₃C—(CH₂)₁₁—C(O)—NH—(CH₂)₃N⁺(CH₃)₂CH₂COO⁻

H₃C—(CH₂)₁₃—C(O)—NH—(CH₂)₃N⁺(CH₃)₂CH₂COO⁻

H₃C—(CH₂)₁₅—C(O)—NH—(CH₂)₃N⁺(CH₃)₂CH₂COO⁻

H₃C—(CH₂)₇—CH═CH—(CH₂)₇—C(O)—NH—(CH₂)₃N⁺(CH₃)₂CH₂COO⁻

Particularly preferably, surfactants of formula (Bet-1) are used within narrower quantity ranges. Hair treatment agents that are preferred here are those that, based on the weight thereof, comprise about 0.25 to about 8 wt. %, more preferably about 0.5 to about 7 wt. %, more preferably about 0.75 to about 6.5 wt. %, and in particular about 1 to about 5.5 wt. % surfactant(s) of formula (Bet-1).

In addition to the amphoteric surfactant or surfactants of formula (Bet-1), or instead of these, the hair treatment agents contemplated herein particularly preferably can comprise, as amphoteric surfactants, betaines of formula (Bet-II)

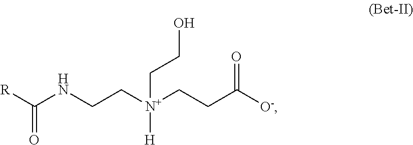

in which R denotes a straight-chain or branched, saturated, monounsaturated or polyunsaturated alkyl or alkenyl group having 8 to 24 carbon atoms.

According to INCI nomenclature, these surfactants are referred to as Amphoacetates, wherein representatives derived from coconut fatty acids are preferred and referred to as Cocoamphoacetates.

For production-related reasons, surfactants of this type always also comprise betaines of formula (Bet-IIa)

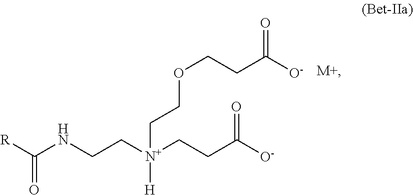

in which R denotes a straight-chain or branched, saturated, monounsaturated or polyunsaturated alkyl or alkenyl group having 8 to 24 carbon atoms, and M denotes a cation.

According to INCI nomenclature, these surfactants are referred to as Amphodiacetates, wherein representatives derived from coconut fatty acids are preferred and referred to as Cocoamphodiacetates.

As contemplated herein, particularly preferably surfactants of formula (Bet-II) that are a mixture of the following representatives are used:

H₃C—(CH₂)₇—C(O)—NH—(CH₂)₂NH⁺(CH₂CH₂OH)CH₂CH₂COO⁻

H₃C—(CH₂)₉—C(O)—NH—(CH₂)₂NH⁺(CH₂CH₂OH)CH₂CH₂COO⁻

H₃C—(CH₂)₁₁—C(O)—NH—(CH₂)₂NH⁺(CH₂CH₂OH)CH₂CH₂COO⁻

H₃C—(CH₂)₁₃—C(O)—NH—(CH₂)₂NH⁺(CH₂CH₂OH)CH₂CH₂COO⁻

H₃C—(CH₂)₁₅—C(O)—NH—(CH₂)₂NH⁺(CH₂CH₂OH)CH₂CH₂COO⁻

H₃C—(CH₂)₇—CH═CH—(CH₂)₇—C(O)—NH—(CH₂)₂NH⁺(CH₂CH₂OH)CH₂CH₂COO⁻

Particularly preferably surfactants of formula (Bet-II) are used within narrower quantity ranges. Hair treatment agents that are preferred here are those that, based on the weight thereof, comprise about 0.25 to about 8 wt. %, more preferably about 0.5 to about 7 wt. %, more preferably about 0.75 to about 6.5 wt. %, and in particular about 1 to about 5.5 wt. % surfactant(s) of formula (Bet-II).

In summary, preferred cosmetic agents contemplated herein are those in which the group R in formulas (Bet-I) and (Bet-II) is selected from

H₃C—(CH₂)₇— H₃C—(CH₂)₉— H₃C—(CH₂)₁₁— H₃C—(CH₂)₁₃— H₃C—(CH₂)₁₅—

H₃C—(CH₂)₇—CH═CH—(CH₂)₇— or mixtures thereof.

The hair treatment agents can additionally comprise non-ionic surfactant(s) and/or cationic surfactant(s).

Suitable non-ionic surfactants include, for example,
addition products of 4 to 30 moles ethylene oxide and/or 0 to 5 moles propylene oxide to linear fatty alcohols having 8 to 22 carbon atoms, to fatty acids having 12 to 22 carbon atoms, and to alkyl phenols having 8 to 15 carbon atoms in the alkyl group;
ethylene oxide and polyglycerol addition products to methyl glucoside fatty acid esters, fatty acid alkanolamides and fatty acid glucamides;
C₈ to C₃₀ fatty acid monoesters and diesters of addition products of 1 to 30 moles ethylene oxide to glycerol;
amine oxides;
sorbitan fatty acid esters and addition products of ethylene oxide to sorbitan fatty acid esters, such as polysorbates;
fatty acid alkanolamides of the following general formula,

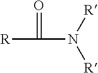

in which R preferably denotes a linear or branched, saturated or unsaturated alkyl or alkenyl group having 8 to 24 carbon atoms, and the groups R′ denote hydrogen or the group —(CH₂)ₙOH, in which n denotes the number 2 or 3, with the proviso that at least one of the groups R′ is the aforementioned group —(CH₂)ₙOH;
sugar fatty acid esters and addition products of ethylene oxide to sugar fatty acid esters;
addition products of ethylene oxide to fatty acid alkanolamides and fatty amines; and/or alkyl(oligo)glucosides;
mixtures of alkyl(oligo)glucosides and fatty alcohols, for example the commercially available product Montanov® 68;
addition products of 5 to 60 moles ethylene oxide to castor oil and hydrogenated castor oil;
partial esters of polyols having 3 to 6 carbon atoms with saturated fatty acids having 8 to 22 carbon atoms;
sterols. Sterols are understood to mean a group of steroids that carry a hydroxyl group at carbon atom 3 of the steroid skeleton and are isolated either from animal tissue (zoosterols) or from vegetable fats (phytosterols). Examples of zoosterols are cholesterol and lanosterol. Examples of suitable phytosterols are ergosterol, stigmasterol and sitosterol. Sterols are also isolated from fungi and yeasts, the so-called mycosterols.
phospholipids. These are understood to mean in particular the glucose phospholipids, which are obtained, for example, as lecithins or phosphatidylcholines from egg yolk or plant seeds, for example (such as soya beans).

Suitable alkyl(oligo)glucosides can be selected from compounds of the general formula RO-[G]ₓ, in which [G] is preferably derived from aldoses and/or ketoses having 5 to 6 carbon atoms, preferably from glucose. The subscript x indicates the degree of oligomerization (DP), which is to say the distribution of monoglycosides and oligoglycosides. The subscript x preferably has a value in the range of 1 to 10, and particularly preferably in the range of 1 to 3, wherein this may not be an integer, but a fractional number, which can be analytically ascertained. Particularly preferred alkyl(oligo)glycosides have a degree of oligomerization between 1.2 to 1.5. The group R preferably denotes at least one alkyl and/or alkenyl group having 4 to 24 carbon atoms. In particular, preferred alkyl(oligo)glycosides are the compounds known by the INCI names Caprylyl/Capryl Glucoside, Decyl Glucoside, Lauryl Glucoside and Coco Glucoside.

Suitable amine oxides can be selected from at least one compound of the general formula (A-I) or (A-II)

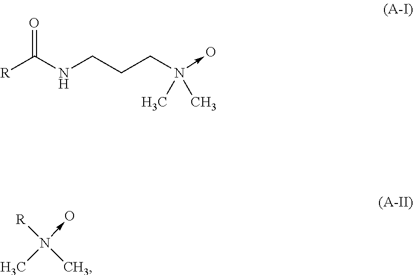

in which R in each case denotes a straight-chain or branched, saturated, monounsaturated or polyunsaturated alkyl or alkenyl group having 6 to 24 carbon atoms, and preferably having 8 to 18 carbon atoms.

Particularly preferred are the surfactants of the above-described formula (A-I) or (A-Ill) known by the INCI names Cocamine Oxide, Lauramine Oxide and/or Cocamidopropylaminoxide, which are commercially available from various suppliers.

Suitable C₈ to C₃₀ fatty acid monoesters and diesters of addition products of 1 to 30 moles ethylene oxide to glycerol shall preferably be understood to mean those by the INCI names PEG (1-10) Glyceryl Cocoate, and in particular PEG-7 Glyceryl Cocoate.

It may furthermore be advantageous to combine the ethoxylated fatty acid esters with further ethoxylated fatty acid esters. Such product mixtures are commercially available, for example from Evonik by the designation “Antil 200®” (INCI name: PEG-200 Hydrogenated Glyceryl Palmate, PEG-7 Glyceryl Cocoate).

Particularly preferred non-ionic surfactants that may be present in the hair treatment agents contemplated herein are fatty acid alkanolamides, in particular the compounds known by the INCI names Cocamide MEA and/or Cocamide MIPA; alkyl(oligo)glucosides, in particular the compounds known by the INCI names Caprylyl/Capryl Glucoside, Decyl Glucoside, Lauryl Glucoside and/or Coco Glucoside; C₈ to C₃₀ fatty acid monoesters and diesters of addition products of 1 to 30 moles ethylene oxide to glycerol, in particular the compound known by the INCI name PEG-7 Glyceryl Cocoate; and/or addition products of 4 to 30 moles ethylene oxide and/or 0 to 5 moles propylene oxide to linear fatty alcohols having 8 to 22 carbon atoms. In particular, Cocamide MEA and/or PEG-7 Glyceryl Cocoate are preferred due to the foam-stabilizing and lipid-replenishing properties thereof.

Cationic surfactants of the quaternary ammonium compound, esterquat and amidoamine types can also be used as contemplated herein. Preferred quaternary ammonium compounds are ammonium halides, in particular chlorides and bromides, such as alkyl trimethylammonium chlorides, dialkyldimethylammonium chlorides and trialkylmethylammonium chlorides. The long alkyl chains of these surfactants preferably comprise 10 to 18 carbon atoms, such as in cetyltrimethylammonium chloride, stearyltrimethylammonium chloride, distearyldimethylammonium chloride, lauryldimethylammonium chloride, lauryldimethyl benzylammonium chloride and tricetylmethylammonium chloride. Further preferred cationic surfactants are the imidazolium compounds known by the INCI names Quaternium-27 and Quaternium-83.

Particularly preferred hair treatment agents as contemplated herein are characterized by comprising, based on the weight thereof, about 0.05 to about 7.5 wt. %, especially about 0.1 to about 5 wt. %, particularly preferably about 0.2 to about 3.5 wt. %, and in particular about 0.25 to about 2.5 wt. % cationic surfactant(s) from the group of quaternary ammonium compounds and/or of the esterquats and/or of the amidoamines as the cationic nourishing substance, wherein preferred cationic surfactant(s) is/are selected from
alkyl trimethylammonium chlorides having preferably 10 to 18 carbon atoms in the alkyl group and/or
dialkyldimethylammonium chlorides having preferably 10 to 18 carbon atoms in the alkyl group and/or
trialkylmethylammonium chlorides having preferably 10 to 18 carbon atoms in the alkyl group and/or
cetyltrimethylammonium chloride and/or
stearyltrimethylammonium chloride and/or
distearyldimethylammonium chloride and/or
lauryldimethylammonium chloride and/or
lauryldimethyl benzylammonium chloride and/or
tricetylmethylammonium chloride
Quaternium-27 and/or
Quaternium-83 and/or
N-methyl-N(2-hydroxyethyl)-N,N-(ditallow acyloxyethyl)ammonium methosulfate and/or
N-methyl-N(2-hydroxyethyl)-N,N-(distearoyloxyethyl)ammonium methosulfate and/or
N,N-dimethyl-N,N-distearoyloxyethyl ammonium chloride and/or
N,N-di-(2-hydroxyethyl)-N,N-(fatty acid ester ethyl) ammonium chloride.

The hair treatment agents comprise at least one divalent or trivalent metal salt. To achieve optimal action, it is advantageous if the metal salts are present in dissolved form in the agents as contemplated herein. In a preferred embodiment, the hair cleaning and care agents contemplated herein thus contain water-soluble divalent or trivalent metal salts. The term “water-soluble” shall be understood to mean that at least 1 g of the particular salt can be completely dissolved in 1 l water at 20° C.

Suitable divalent or trivalent metal salts may be selected from divalent or trivalent organic and/or inorganic salts. If formates are used, these must be assigned to component f) (compounds of formula (I)) and are not considered in the calculation of component g) (divalent or trivalent metal salt).

Particularly suitable cations within these salts can preferably be selected from alkaline earth metal cations, as well as copper, zinc, iron(II), iron(III) and/or aluminum cations.

Alkaline earth metal cations are especially particularly preferred, and in particular calcium and magnesium cations are preferred. Particularly suitable organic anions within these salts can particularly be selected from acetate, lactate, succinate, citrate, tartrate, malate, maleate, oxalate and/or glycolate ions. Especially particularly preferred are acetate, lactate and/or citrate salts comprising the aforementioned cations. In particular, preferred organic salts are calcium lactate, calcium citrate, calcium acetate, magnesium lactate, magnesium citrate and/or magnesium acetate. Particularly suitable inorganic anions within these salts may be selected from halide, sulfate, phosphate and/or carbonate ions. Sulfate and/or halide ions are especially particularly preferred, such as chloride and bromide ions. In particular, preferred inorganic salts are calcium chloride, calcium sulfate, magnesium chloride and/or magnesium sulfate.

The percentage of weight of the at least one divalent or trivalent metal salt based on the total weight of the hair treatment agent as contemplated herein is preferably about 0.01 to about 10 wt. %, especially about 0.1 to about 7.5 wt. %, more preferably about 0.2 to about 5 wt. %, and in particular about 0.3 to about 3 wt. %.

Preferred hair treatment agents as contemplated herein comprise, based on the weight thereof, about 0.01 to about 10 wt. %, especially about 0.1 to about 7.5 wt. %, more preferably about 0.2 to about 5 wt. %, and in particular about 0.3 to about 3 wt. % of at least one divalent or trivalent metal salt from the group of the organic or inorganic copper, zinc, iron(II), calcium, magnesium, iron(III) and/or aluminum salts.

Within this embodiment, the water-soluble salts are particularly preferred. Especially particularly preferred within this embodiment are calcium lactate, calcium citrate, calcium acetate, magnesium lactate, magnesium citrate, magnesium acetate, calcium halide, calcium hydroxide, magnesium halide and/or magnesium hydroxide.

The hair treatment agents comprise at least one divalent or trivalent metal salt. To achieve optimal action, it is advantageous if the metal salts are present in dissolved form in the agents contemplated herein. In a preferred embodiment, the hair cleaning and care agents contemplated herein thus contain water-soluble divalent or trivalent metal salts. The term “water-soluble” shall be understood to mean that at least 1 g of the particular salt can be completely dissolved in 1 l water at 20° C.

Suitable divalent or trivalent metal salts may be selected from divalent or trivalent organic and/or inorganic salts. Particularly suitable cations within these salts can preferably be selected from alkaline earth metal cations, as well as copper, zinc, iron(II), iron(III) and/or aluminum cations. Alkaline earth metal cations are especially particularly preferred, and in particular calcium and magnesium cations are preferred. Particularly suitable organic anions within these salts can particularly be selected from acetate, lactate, succinate, citrate, tartrate, malate, maleate, oxalate and/or glycolate ions. Especially particularly preferred are acetate, lactate and/or citrate salts comprising the aforementioned cations. In particular, preferred organic salts are calcium lactate, calcium citrate, calcium acetate, magnesium lactate, magnesium citrate and/or magnesium acetate. Particularly suitable inorganic anions within these salts may be selected from halide, sulfate, phosphate and/or carbonate ions. Sulfate and/or halide ions are especially particularly preferred, such as chloride and bromide ions. In particular, preferred inorganic salts are calcium chloride, calcium sulfate, magnesium chloride and/or magnesium sulfate.

The percentage of weight of the at least one divalent or trivalent metal salt based on the total weight of the hair treatment agent contemplated herein is preferably about 0.01 to about 10 wt. %, especially about 0.1 to about 7.5 wt. %, more preferably about 0.2 to about 5 wt. %, and in particular about 0.3 to about 3 wt. %.

Preferred hair treatment agents as contemplated herein comprise, based on the weight thereof, about 0.01 to about 10 wt. %, especially about 0.1 to about 7.5 wt. %, more preferably about 0.2 to about 5 wt. %, and in particular about 0.3 to about 3 wt. % of at least one divalent or trivalent metal salt from the group of the organic or inorganic copper, zinc, iron(II), calcium, magnesium, iron(III) and/or aluminum salts.

Within this embodiment, the water-soluble salts are particularly preferred. Especially particularly preferred within this embodiment are calcium lactate, calcium citrate, calcium acetate, magnesium lactate, magnesium citrate, magnesium acetate, calcium halide, calcium hydroxide, magnesium halide and/or magnesium hydroxide.

The hair treatment agents comprise at least one organic acid. Typical representatives for organic acids are aliphatic monocarboxylic and dicarboxylic acids, such as acetic acid, propionic acid, oxalic acid, and 1,3-propanedioic acid, as well as aromatic carboxylic acids, such as benzoic acid. Further organic acids are, for example, hydroxycarboxylic acids such as glycolic acid, citric acid, tartaric acid, malic acid and lactic acid. Unsaturated monocarboxylic or dicarboxylic acids, such as fumaric acid, or α-keto carboxylic acids, such as pyruvic acid (2-oxopropionic acid) are also contemplated herein.

Preferred hair treatment agents as contemplated herein comprise, based on the weight thereof, about 0.001 to about 10 wt. %, especially about 0.01 to about 5 wt. %, more preferably about 0.05 to about 3 wt. %, and in particular about 0.1 to about 2 wt. % organic acid(s).

Particularly preferred hair treatment agents comprise, based on the weight thereof, about 0.001 to about 10 wt. %, especially about 0.01 to about 5 wt. %, more preferably about 0.05 to about 3 wt. %, and in particular about 0.1 to about 2 wt. % organic acid(s) from the group consisting of formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluoyl acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrole carboxylic acid, 1,2,4,6,7-napthalene pentaacetic acid, malonaldehyde acid, 4-hydroxyphthalimide acid, 1-pyrazole carboxylic acid, gallic acid or propanetricarboxylic acid, glycolic acid, lactic acid, malic acid, tartaric acid or citric acid.

Several representatives among the aforementioned acids are particularly preferred since these additionally impart a pleasant shine to the hair treated with the agents contemplated herein. Exceptionally preferred hair treatment agents contemplated herein comprise, based on the weight thereof, about 0.001 to about 10 wt. %, especially about 0.01 to about 5 wt. %, more preferably about 0.05 to about 3 wt. %, and in particular about 0.1 to about 2 wt. % organic acid(s) from the group consisting of formic acid, oxalic acid, maleic acid, lactic acid, tartaric acid or citric acid.

The agents as contemplated herein comprise ethyl lauroyl arginate (ELA or LAE)

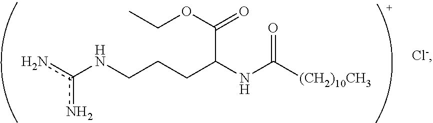

which is also referred to as ethyl-N^{alpha}-dodecanoyl-L-arginate hydrochloride. LAE is commercially available and considered to be safe, also for use in foods.

Preferred hair treatment agents as contemplated herein comprise, based on the weight thereof, about 0.001 to about 2 wt. %, especially about 0.01 to about 1 wt. %, more preferably about 0.025 to about 0.6 wt. %, and in particular about 0.05 to about 0.4 wt. % ethyl lauroyl arginate HCl.

The hair treatment agents preferably comprise the above-described active ingredients in a cosmetically acceptable carrier. Within the scope of the invention, this is preferably understood to mean an aqueous or aqueous-alcoholic carrier.

The cosmetic carrier preferably contains at least about 50 wt. %, more preferably at least about 60 wt. %, particularly preferably at least about 70 wt. %, and in particular preferably at least about 75 wt. % water.

Furthermore, the cosmetic carrier can comprise about 0.01 to about 40 wt. %, preferably about 0.05 to about 30 wt. %, and in particular about 0.1 to about 20 wt. % of at least one alcohol.

Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, or mixtures of these alcohols. The water-soluble alcohols are particularly preferred. In particular, ethanol, 1,2-propylene glycol, glycerol, benzyl alcohol and mixtures of these alcohols are preferred.

For excellent skin (scalp) tolerability of the hair treatment agents contemplated herein, it is advantageous if these have a slightly acidic pH value. It was found that the agents as contemplated herein offer particularly good skin tolerability and mildness in a pH range from about 4.2 to about 5.8. In a first preferred embodiment, the hair treatment agents as contemplated herein thus preferably have a pH value in the range of about 4.2 to about 5.8, more preferably of about 4.25 to about 5.6, particularly preferably of about 4.3 to about 5.5, exceptionally preferably of about 4.35 to about 5.4, and in particular preferably of about 4.4 to about 5.3.

The agents contemplated herein can comprise cationic polymer(s).

Regardless of which cationic polymer(s) is/are used, preferred hair treatment agents comprise, based on the weight of the agent, about 0.01 to about 3 wt. %, especially about 0.05 to about 2 wt. %, more preferably about 0.1 to about 1.5 wt. %, and in particular about 0.15 to about 0.8 wt. % cationic polymer(s).

Cationic polymers that can preferably be used as contemplated herein are described hereafter:

Homopolymers of general formula (G1-I)

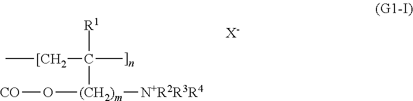

in which R¹=—H or —CH₃, R², R³ and R⁴, independently of one another, are selected from C₁₋₄ alkyl, -alkenyl or -hydroxyalkyl groups, m=1, 2, 3 or 4, n is a natural number, and X⁻ is a physiologically compatible organic or inorganic anion, and copolymers consisting essentially of the monomer units listed in formula (G1-I), and nonionogenic monomer units, are particularly preferred cationic polymers. Among these polymers, those to which at least one of the following conditions applies are preferred:
R¹ denotes a methyl group;
R², R³ and R⁴ denote methyl groups;
m has the value 2.

Suitable physiologically compatible counterions X are, for example, halide ions, sulfate ions, phosphate ions, methosulfate ions, and organic ions, such as lactate, citrate, tartrate and acetate ions. Halide ions, and in particular chloride, are preferred.

A particularly suitable homopolymer is the, optionally cross-linked, poly(methacryloyloxyethyltrimethylammonium chloride) by the INCI name Polyquaternium-37. Such products are commercially available, for example, under the designations Rheocare®CTH (Cosmetic Rheologies) and Synthalen® CR (Ethnichem). If desired, the crosslinking can take place with the help of polyolefinically unsaturated compounds, for example divinylbenzene, tetraallyloxyethane, methylenebisacrylamide, diallyl ether, polyallyl polyglyceryl ether, or allyl ethers of sugars or sugar derivatives, such as erythritol, pentaerythritol, arabitol, mannitol, sorbitol, sucrose or glucose. Methylenebisacrylamide is a preferred crosslinking agent.

The homopolymer is preferably used in the form of a non-aqueous polymer dispersion, which should have a polymer content of no less than 30 wt. %. Such polymer dispersions are commercially available under the names Salcare® SC 95 (approximately 50% polymer content, further components: mineral oil (INCI name: Mineral Oil) and tridecyl polyoxypropylene polyoxyethylene ether (INCI name: PPG-1-Trideceth-6)), and Salcare® SC 96 (approximately 50% polymer content, further components: mixture of diesters of propylene glycol with a mixture of caprylic acid and capric acid (INCI name: Propylene Glycol Dicaprylate/Dicaprate) and tridecyl polyoxypropylene polyoxyethylene ether (INCI name: PPG-1-Trideceth-6)).

Copolymers including monomer units according to formula (G1-I) preferably comprise acrylamide, methacrylamide, C₁₋₄ alkyl acrylates and C₁₋₄ alkyl methacrylates as nonionogenic monomer units. Among these nonionogenic monomers, acrylamide is particularly preferred. As in the case of the homopolymers described above, these copolymers may also be crosslinked. A copolymer that is preferred as contemplated herein is the crosslinked acrylamide-methacryloyloxyethyltrimethylammonium chloride copolymer. Such copolymers in which the monomers are present in a weight ratio of approximately 20:80 are commercially available as an approximately 50% non-aqueous polymer dispersion under the designation Salcare® SC 92.

Further preferred cationic polymers are, for example:
quaternized cellulose derivatives, as they are commercially available under the designations Celquat® and Polymer JR®. The compounds Celquat® H 100, Celquat® L 200 and Polymer JR®400 are preferred quaternized cellulose derivatives;
cationic alkyl polyglycosides;
cationized honey, for example the commercial product Honeyquat® 50;
cationic guar derivatives, such as in particular the products sold under the trade names Cosmedia® Guar and Jaguar®;
polymeric dimethyl diallyl ammonium salts and the copolymers thereof with esters and amides of acrylic acid and methacrylic acid. The products available commercially under the designations Merquat® 100 (poly(dimethyldiallylammonium chloride)) and Merquat® 550 (dimethyldiallylammonium chloride/acrylamide copolymer) are examples of such cationic polymers;
copolymers of vinylpyrrolidone with quaternized derivatives of dialkyl aminoalkyl acrylate and methacrylate, such as diethyl sulfate-quaternized vinylpyrrolidone/dimethylaminoethyl methacrylate copolymers. Such compounds are commercially available under the designations Gafquat®734 and Gafquat® 755;
vinylpyrrolidone/vinylimidazolium methochloride copolymers, as they are offered under the designations Luviquat® FC 370, FC 550, FC 905 and HM 552;
quaternized polyvinyl alcohol;
and the polymers known under the designations Polyquaternium-2, Polyquaternium-17, Polyquaternium-18 and Polyquaternium-27, comprising quaternary nitrogen atoms in the polymer main chain.

It is likewise possible to use the polymers known under the designations Polyquaternium-24 (commercial product, such as Quatrisoft® LM 200) as cationic polymers. According to the invention, it is likewise possible to use the copolymers of vinylpyrrolidone, as they are available as commercial products Copolymer 845 (manufacturer: ISP), Gaffix® VC 713 (manufacturer: ISP), Gafquat®ASCP 1011, Gafquat®HS 110, Luviquat®8155 and Luviquat® MS 370.

It is also possible to use cationic protein hydrolysates as cationic polymers, wherein preferred agents comprise one or more cationic protein hydrolysates from the group consisting of Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Soy Protein and Quaternium-79 Hydrolyzed Wheat Protein.

It is particularly preferred if cationic polysaccharide polymers are used herein as cationic polymers.

Cationic polysaccharide polymers enhance the care performance of the hair treatment agents contemplated herein (in particular the effectiveness of the agents contemplated herein against hair breakage). Suitable cationic polysaccharide polymers can be selected from cationic cellulose compounds and/or from cationic guar derivatives.

Particularly preferred hair treatment agents as contemplated herein comprise, based on the weight of the agent, about 0.01 to about 3 wt. %, especially about 0.05 to about 2 wt. %, more preferably about 0.1 to about 1.5 wt. %, and in particular about 0.15 to about 0.8 wt. % of at least one polymer from the group of cationic cellulose polymers and/or of the cationic guar derivatives as the cationic polysaccharide polymer(s).

Cationic cellulose compounds within the meaning of the invention are those that carry more than one permanent cationic charge in at least one side chain. Cellulose is composed of D-glucopyranose units linked by beta-1,4-glycosidic bonds and forms unbranched, water-insoluble chains. A “side chain” of cellulose is defined as being chemical substituents that bind to the cellulose skeleton and are not counted as part of the native cellulose since they are subsequently introduced, such as by way of chemical synthesis.

Preferred are quaternized cellulose polymers based on hydroxy(C₂-C₄)alkyl celluloses, and particularly preferably based on hydroxyethyl celluloses. These polymers are known to a person skilled in the art and commercially available from a variety of firms. Particularly preferred are the cationic cellulose derivatives known by the INCI names Polyquaternium-4, Polyquaternium-10, Polyquaternium-24, Polyquaternium-67 and/or Polyquaternium-72. Especially particularly preferred are Polyquaternium-10, Polyquaternium-24 and/or Polyquaternium-67, in particular Polyquaternium-10 being preferred.

Preferred hair treatment agents as contemplated herein comprise, based on the weight of the agent, about 0.01 to about 3 wt. %, especially about 0.05 to about 2 wt. %, more preferably about 0.1 to about 1.5 wt. %, and in particular about 0.15 to about 0.8 wt. % of at least one polymer from the group of Polyquaternium-4, Polyquaternium-10, Polyquaternium-24, Polyquaternium-67 and/or Polyquaternium-72 as the cationic polysaccharide polymer(s).

Particularly preferred hair treatment agents as contemplated herein comprise, based on the weight of the agent, about 0.01 to about 3 wt. %, especially about 0.05 to about 2 wt. %, more preferably about 0.1 to about 1.5 wt. %, and in particular about 0.15 to about 0.8 wt. % Polyquaternium-10 as the cationic polysaccharide polymer(s).

Suitable cationic guar derivatives as contemplated herein are cationic hydroxyalkyl guar derivatives, especially cationic hydroxyethyl trimethylammonium guar and/or cationic hydroxypropyl trimethylammonium guar having average molecular weights between 100,000 and 2,000,000 daltons. In particular, the cationic guar polymers having a molecular weight (weight average molecular weight) between 200,000 and 1,600,000 daltons known by the INCI name Guar Hydroxypropyltrimonium Chloride are preferred. The cationic charge density of these guar polymers is especially at least about 0.4 meq/g, preferably at least about 0.5 meq/g, and in particular at least about 0.6 meq/g. The nitrogen content is especially in the range of about 1.1 to about 1.8 wt. % (based on the total weight). Cationic guar derivatives known by the INCI name Guar Hydroxypropyltrimonium Chloride are known to a person skilled in the art and available from a variety of suppliers, for example under the trade names Cosmedia® Guar, N-Hance® and/or Jaguar®.

Particularly preferred hair treatment agents comprise, based on the weight of the agent, about 0.01 to about 3 wt. %, especially about 0.05 to about 2 wt. %, more preferably about 0.1 to about 1.5 wt. %, and in particular about 0.15 to about 0.8 wt. % Guar Hydroxypropyltrimonium Chloride as the cationic polysaccharide polymer(s).

The hair treatment agents contemplated herein can comprise vegetable oils, vegetable butters and/or vegetable waxes. These vegetable oil components impart improved combing and styling properties to the hair and increased shine.

Suitable vegetable oil components include natural (vegetable) oils and/or butters, which typically comprise triglycerides and mixtures of triglycerides. Preferred natural oils are coconut oil, (sweet) almond oil, walnut oil, peach kernel oil, apricot kernel oil, argan oil, avocado oil, tea tree oil, soy bean oil, sesame oil, sunflower oil, tsubaki oil, evening primrose oil, rice bran oil, palm kernel oil, mango kernel oil, marula oil, lady's smock oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter and/or shea butter.

Suitable natural or vegetable waxes that can preferably be used are carnauba waxes, beeswaxes and/or candelilla waxes.

Particularly preferred vegetable oil components are (sweet) almond oil, peach kernel oil, apricot kernel oil, amaranth seed oil, argan oil, olive oil, jojoba oil, cocoa butter and/or shea butter. In particular, apricot kernel oil, argan oil, olive oil and/or jojoba oil are preferred.

In a preferred embodiment, the hair treatment agents as contemplated herein preferably comprise coconut oil, (sweet) almond oil, walnut oil, peach kernel oil, apricot kernel oil, argan oil, avocado oil, tea tree oil, soy bean oil, sesame oil, sunflower oil, tsubaki oil, evening primrose oil, rice bran oil, palm kernel oil, mango kernel oil, marula oil, lady's smock oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter and/or shea butter.

Within this embodiment, it is particularly preferred if the hair treatment agents contemplated herein comprise (sweet) almond oil, peach kernel oil, apricot kernel oil, amaranth seed oil, argan oil, olive oil, jojoba oil, cocoa butter and/or shea butter.

The percentage of weight of the at least one vegetable oil, vegetable butter and/or vegetable wax based on the total weight of the hair treatment agent contemplated herein is preferably about 0.02 to about 2.50 wt. %, more preferably about 0.03 to about 2.00 wt. %, particularly preferably about 0.04 to about 1.50 wt. %, and in particular about 0.05 to about 1.00 wt. %.

In addition to the above-mentioned essential and optional components, in one further preferred embodiment the hair treatment agents contemplated herein may include at least one further hair conditioning active ingredient so as to further enhance the nourishing properties of the agents, which may be selected from the group consisting of:
protein hydrolysates;
vitamins;
plant extracts and/or
glycerol.

Suitable protein hydrolysates shall be understood to mean product mixtures that can be obtained by the acidically, basically or enzymatically catalyzed degradation of proteins. Protein hydrolysates of plant, animal and/or marine origin can be used.

Animal protein hydrolysates are, for example, elastin, collagen, keratin, silk and milk protein hydrolysates, which can also be present in the form of salts. Such products are sold, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Protein hydrolysates of plant origin, such as soy, almond, rice, pea, potato and wheat protein hydrolysates, are preferred. Such products are available, for example, under the trademarks Gluadin® (Cognis), DiaMin® (Diamalt), Lexein® (Inolex) and Crotein® (Croda). It is also possible to use cationized protein hydrolysates, wherein the underlying protein hydrolysate can stem from animals, for example from collagen, milk or keratin, from plants, for example from wheat, maize, rice, potatoes, soy or almonds, from marine life forms, for example from fish collagen or algae, or from protein hydrolysates obtained by way of biotechnology. The protein hydrolysates underlying the cationic derivatives can be obtained from the corresponding proteins by way of chemical, in particular alkaline or acid hydrolysis, enzymatic hydrolysis and/or a combination of the two types of hydrolysis. The hydrolysis of proteins generally results in a protein hydrolysate having a molecular weight distribution from approximately 100 daltons to several thousand daltons Cationic protein hydrolysates in which the underlying protein component has a molecular weight of 100 to 25,000 daltons, and preferably 250 to 5000 daltons, are preferred. Moreover, cationic protein hydrolysates shall be understood to mean quaternized amino acids and the mixtures thereof. The quaternization of the protein hydrolysates or of the amino acids is frequently carried out using quaternary ammonium salts, such as N,N-dimethyl-N-(n-alkyl)-N-(2-hydroxy-3-chloro-n-propyl) ammonium halides. The cationic protein hydrolysates can moreover also be further derivatized. Typical examples of the cationic protein hydrolysates and derivatives that shall be mentioned are the commercially available products known under the following INCI names: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

The percentage by weight of the protein hydrolysate or protein hydrolysates based on the total weight of the hair treatment agents is preferably about 0.01 to about 5 wt. %, more preferably about 0.025 to about 3 wt. %, and in particular about 0.05 to about 2 wt. %.

Regardless of the source (vegetable, animal, maritime and the like), protein hydrolysates comprise individual amino acids, oligopeptides and optionally polypeptides, depending on the degree of hydrolysis.

It is particularly preferred if the hair treatment agents as contemplated herein comprise at least one oligopeptide including at least one amino acid sequence Glu-Glu-Glu

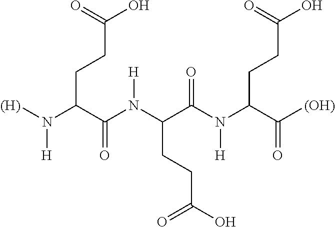

wherein the amino group may be present in a free or protonated state, and the carboxy groups may be present in a free or deprotonated state.

Preferred hair treatment agents contemplated herein are characterized by comprising, based on the weight thereof, about 0.0001 to about 10 wt. % of at least one oligopeptide including at least one amino acid sequence Glu-Glu-Glu

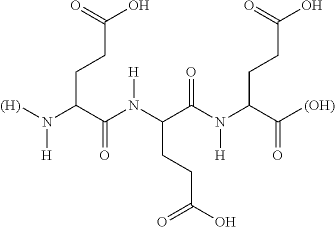

wherein the amino group may be present in a free or protonated state, and the carboxy groups may be present in a free or deprotonated state.

In this formula, as in all formulas hereafter, the bracketed hydrogen atom of the amino group as well as the bracketed hydroxy group of the acid function mean that the groups in question may be present per se (this involves an oligopeptide having the relevant number of amino acids, as shown) (3 in the formula above), or else that the amino acid sequence is present in an oligopeptide that includes further amino acids; depending on where the further amino acid or amino acids is or are bound, the bracketed components of the above-mentioned formula are replaced by the further amino acid group(s).

These preferred hair treatment agents as contemplated herein comprise, based on the weight thereof, about 0.0001 to about 10 wt. %, of at least one oligopeptide including at least one amino acid sequence Glu-Glu-Glu, which is to say at least three consecutive glutamic acids.

Oligopeptides within the meaning of the present application are condensation products of amino acids comprising at least 3 and no more than 25 amino acids, the condensation products being linked by peptide bonds in an acid amide-like manner. In preferred hair treatment agents as contemplated herein, the oligopeptide comprises 5 to 15 amino acids, especially 6 to 13 amino acids, particularly preferably 7 to 12 amino acids, and in particular 8, 9 or 10 amino acids.

Depending on whether further amino acids are bound to the Glu-Glu-Glu sequence, and depending on the type of these amino acids, the molar mass of the oligopeptide present in the agents may vary. Preferred hair treatment agents as contemplated herein are characterized in that the oligopeptide has a molar mass of 650 to 3000 Da, especially of 750 to 2500 Da, particularly preferably of 850 to 2000 Da, and in particular of 1000 to 1600 Da.

In summary, preferred hair treatment agents are characterized in that the oligopeptide comprises 5 to 15 amino acids, especially 6 to 13 amino acids, particularly preferably 7 to 12 amino acids, and in particular 8, 9 or 10 amino acids, and has a molar mass of 650 to 3000 Da, especially of 750 to 2500 Da, particularly preferably of 850 to 2000 Da, and in particular of 1000 to 1600 Da.

As is apparent from the preferred number of amino acids in the oligopeptides and the preferred molar mass range, oligopeptides that are not composed solely of the three glutamic acids, but comprise further amino acids bound to this sequence are preferred. These further amino acids are preferably selected from certain amino acids, while certain other representatives are less preferred.

It is preferred, for example, if the oligopeptides used in the agents contemplated herein do not contain any methionine.

It is further preferred if the oligopeptides used in the agents contemplated herein do not contain any cysteine and/or cystine.

It is further preferred if the oligopeptides used in the agents contemplated herein do not contain any aspartic acid and/or asparagine.

It is further preferred if the oligopeptides used in the agents contemplated herein do not contain any serine and/or threonine.

In contrast, it is preferred if the oligopeptides used in the agents contemplated contain tyrosine.

It is further preferred if the oligopeptides used in the agents contemplated herein contain leucine.

It is further preferred if the oligopeptides used in the agents contemplated herein contain isoleucine.

It is further preferred if the oligopeptides used in the agents contemplated herein contain arginine.

It is further preferred if the oligopeptides used in the agents contemplated herein contain valine.

Particularly preferred oligopeptides, or amino acid sequences present in the preferred oligopeptides, are described hereafter.

A particularly preferred oligopeptide additionally comprises tyrosine, which is preferably bound to the Glu-Glu-Glu sequence via the acid function. Preferred hair treatment agents contemplated herein are thus characterized in that the oligopeptide present therein includes at least one amino acid sequence Tyr-Glu-Glu-Glu

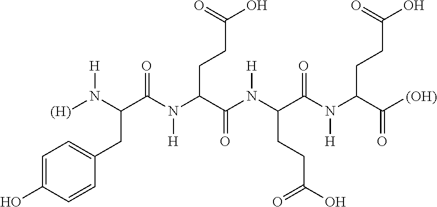

wherein the amino group may be present in a free or protonated state, and the carboxy groups may be present in a free or deprotonated state.

A further particularly preferred oligopeptide additionally comprises isoleucine, which is preferably bound to the Glu-Glu-Glu sequence via the amino function. Preferred hair treatment agents are thus characterized in that the oligopeptide present therein includes at least one amino acid sequence Glu-Glu-Glu-Ile

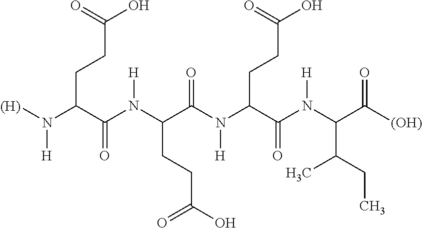

wherein the amino group may be present in a free or protonated state, and the carboxy groups may be present in a free or deprotonated state.

Oligopeptides that include both of the above-mentioned amino acids (tyrosine and isoleucine) are preferred. Particularly preferred are hair treatment agents contemplated herein in which the oligopeptide present in the hair treatment agent includes at least one amino acid sequence Tyr-Glu-Glu-Glu-Ile

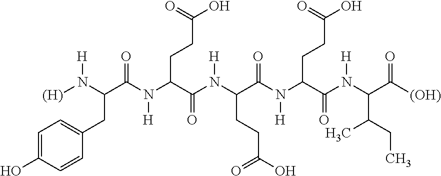

wherein the amino group may be present in a free or protonated state, and the carboxy groups may be present in a free or deprotonated state.

Further preferred oligopeptides additionally comprise arginine, which is preferably present bound to isoleucine.

Particularly preferred are hair treatment agents contemplated herein in which the oligopeptide present in the hair treatment agent includes at least one amino acid sequence Tyr-Glu-Glu-Glu-Ile-Arg

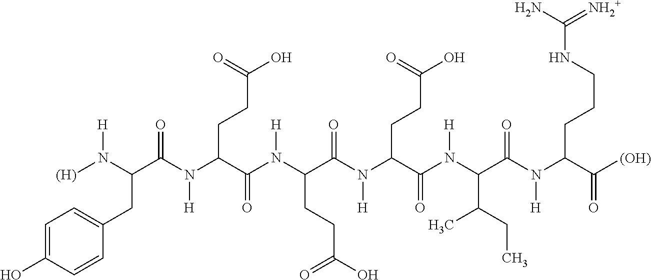

wherein the amino groups may be present in a free or protonated state, and the carboxy groups may be present in a free or deprotonated state.

Still further preferred oligopeptides additionally comprise valine, which is preferably present bound to the arginine. Further preferred hair treatment agents contemplated herein are thus characterized in that the oligopeptide present in the hair treatment agent includes at least one amino acid sequence Tyr-Glu-Glu-Glu-Ile-Arg-Val

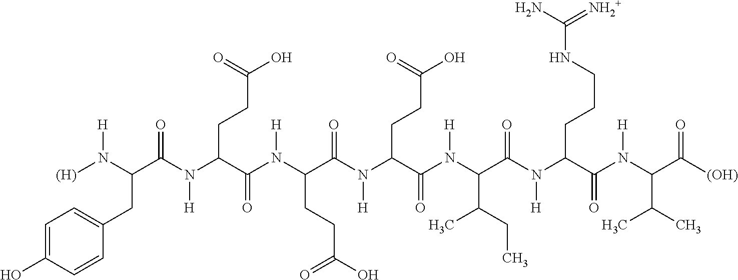

wherein the amino groups may be present in a free or protonated state, and the carboxy groups may be present in a free or deprotonated state.

Still further preferred oligopeptides additionally comprise leucine, which is preferably present bound to the valine. Further preferred hair treatment agents as contemplated herein are thus characterized in that the oligopeptide present in the hair treatment agent includes at least one amino acid sequence Tyr-Glu-Glu-Glu-Ile-Arg-Val-Leu

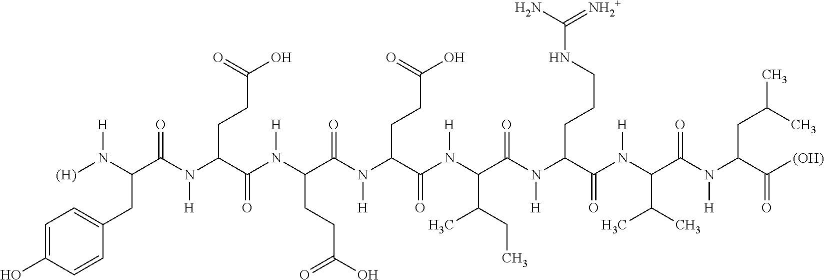

wherein the amino groups may be present in a free or protonated state, and the carboxy groups may be present in a free or deprotonated state.

In particular, preferred oligopeptides additionally comprise leucine, which is preferably present bound to the tyrosine. Further preferred hair treatment agents as contemplated herein are thus characterized in that the oligopeptide present in the hair treatment agent includes at least one amino acid sequence Leu-Tyr-Glu-Glu-Glu-Ile-Arg-Val-Leu

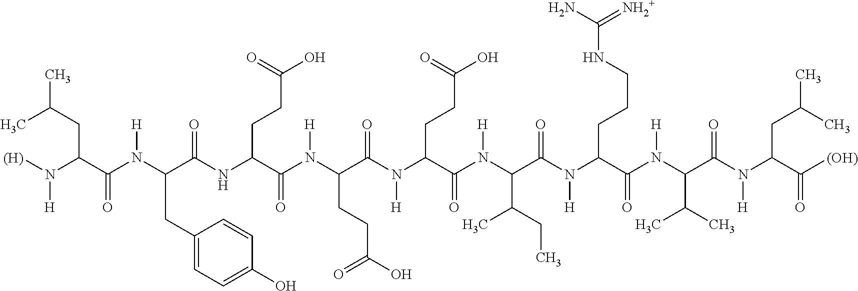

wherein the amino groups may be present in a free or protonated state, and the carboxy groups may be present in a free or deprotonated state.

It is especially particularly preferred if agents contemplated herein comprise at least two oligopeptides that satisfy the above-described criteria, but differ from one another. For example, the use of hair treatment agents comprising at least two oligopeptides A and B that are different from one another, but both include the amino acid sequence Glu-Glu-Glu, is preferred.

These oligopeptides A and B that are different from one another correspond to each other in that these carry three consecutive Glu amino acids in the amino acid sequence, while they have differences in the amino acids bound before or after that. Preferred are peptides that are different from one another and exhibit partial agreement with each other, which may also be greater than the above-mentioned three amino acids.

For example, further preferred hair treatment agents are characterized in that at least two oligopeptides A and B that are different from one another, but both include the amino acid sequence Glu-Glu-Glu-Ile, are present in the hair treatment agent.

Hair treatment agents comprising at least two oligopeptides A and B that are different from one another, but both include the amino acid sequence Ty-Glu-Glu-Glu, are likewise preferred.

Still further preferred hair treatment agents are characterized by comprising at least two oligopeptides A and B that are different from one another, but both include the amino acid sequence Glu-Glu-Glu-Ile-Arg.

Likewise, still further preferred hair treatment agents are characterized in that at least two oligopeptides A and B that are different from one another, but both include the amino acid sequence Tyr-Glu-Glu-Glu-Ile are present in the hair treatment agent.

Preferred hair treatment agents as contemplated herein are thus characterized in that the oligopeptide includes at least one amino acid sequence Tyr-Glu-Glu-Glu-Ile

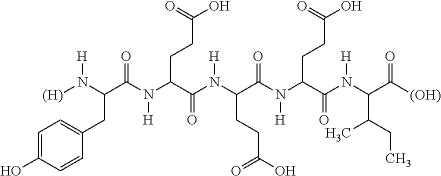

wherein the amino group may be present in a free or protonated state, and the carboxy groups may be present in a free or deprotonated state.

Especially particularly preferred hair treatment agents are characterized in that at least two oligopeptides A and B that are different from one another, but both include the amino acid sequence Glu-Glu-Glu-Ile-Arg are present in the hair treatment agent

Likewise, especially particularly preferred hair treatment agents are characterized in that at least two oligopeptides A and B that are different from one another, but both include the amino acid sequence Tyr-Glu-Glu-Glu-Ile-Arg are present in the hair treatment agent

The oligopeptides preferably have an even greater structural agreement. For example, hair treatment agents comprising at least two oligopeptides A and B that are different from one another, but both include the amino acid sequence Glu-Glu-Glu-Ile-Arg-Val, are further preferred embodiments of the present invention.

Hair treatment agents comprising at least two oligopeptides A and B that are different from one another, but both include the amino acid sequence Tyr-Glu-Glu-Glu-Ile-Arg-Val, are likewise preferred embodiments.

Still further preferred hair treatment agents contemplated herein are characterized by comprising at least two oligopeptides A and B that are different from one another, but both include the amino acid sequence Glu-Glu-Glu-Ile-Arg-Val-Leu.

Likewise, still further preferred hair treatment agents contemplated herein are characterized by comprising at least two oligopeptides A and B that are different from one another, but both include the amino acid sequence Tyr-Glu-Glu-Glu-Ile-Arg-Val-Leu.

Preferred hair treatment agents contemplated herein are thus characterized in that the oligopeptide includes at least one amino acid sequence Tyr-Glu-Glu-Glu-Ile-Arg-Val-Leu

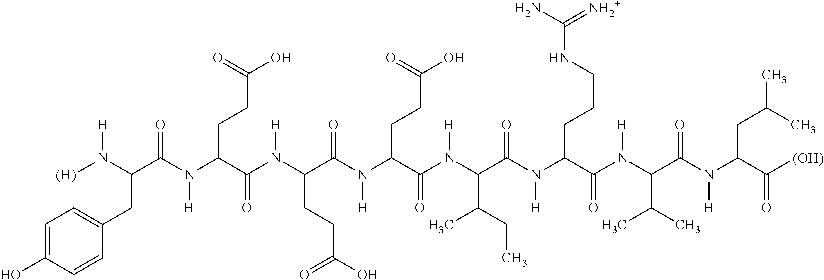

wherein the amino groups may be present in a free or protonated state, and the carboxy groups may be present in a free or deprotonated state.

In particular, preferred hair treatment agents contemplated herein are characterized by comprising at least two oligopeptides A and B that are different from one another, wherein oligopeptide A includes the amino acid sequence Leu-Tyr-Glu-Glu-Glu-Ile-Arg-Val-Leu

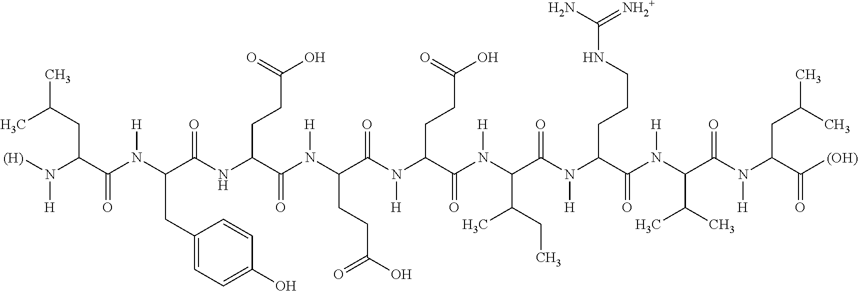

wherein the amino groups may be present in a free or protonated state, and the carboxy groups may be present in a free or deprotonated state, and oligopeptide B includes the amino acid sequence Tyr-Glu-Glu-Glu-Ile-Arg-Val-Leu

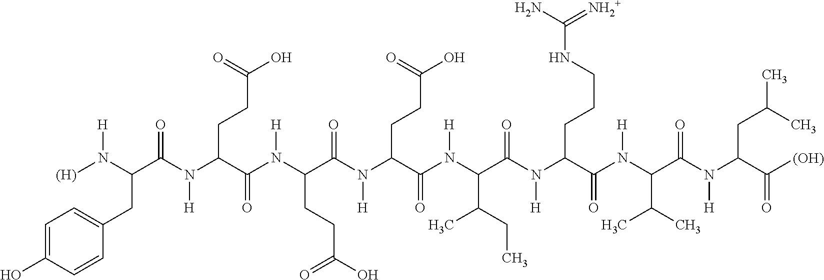

wherein the amino groups may be present in a free or protonated state, and the carboxy groups may be present in a free or deprotonated state.

In especially particularly preferred hair treatment agents of this last embodiment, these comprise, based on the weight of the agent, about 0.00001 to about 1 wt. % oligopeptide A and about 0.00001 to about 1 wt. % oligopeptide B.

In further preferred hair treatment agents of this last embodiment, these comprise, based on the weight of the agent, about 0.00005 to about 0.1 wt. % oligopeptide A and about 0.00005 to about 0.1 wt. % oligopeptide B.

In still further preferred hair treatment agents of this last embodiment, these comprise, based on the weight of the agent, about 0.0001 to about 0.01 wt. % oligopeptide A and about 0.0001 to about 0.001 wt. % oligopeptide B.

The oligopeptides that are used as contemplated herein and satisfy the above-described conditions can advantageously be obtained from keratinic materials. It is preferred that these oligopeptides are used in high proportions, based on the entire keratinic peptide content of the agents.

It is especially particularly preferred that a preferably high proportion of all keratinic peptides present in the agent contemplated herein satisfies the above-described conditions.

Preferred hair treatment agents contemplated herein are characterized in that at least about 0.1 wt. %, especially at least about 0.5 wt. %, particularly preferably at least about 1 wt. %, more preferably at least about 2.5 wt. %, still more preferably at least about 5 wt. %, and in particular at least about 10 wt. % of all keratinic peptides present in the agent include the amino acid sequence Glu-Glu-Glu.

Further preferred hair treatment agents contemplated herein are characterized in that at least about 0.1 wt. %, especially at least about 0.5 wt. %, particularly preferably at least about 1 wt. %, more preferably at least about 2.5 wt. %, still more preferably at least about 5 wt. %, and in particular at least about 10 wt. % of all keratinic peptides present in the agent include the amino acid sequence Glu-Glu-Glu-Ile.

Still further preferred hair treatment agents contemplated herein are characterized in that at least about 0.1 wt. %, especially at least about 0.5 wt. %, particularly preferably at least about 1 wt. %, more preferably at least about 2.5 wt. %, still more preferably at least about 5 wt. %, and in particular at least about 10 wt. % of all keratinic peptides present in the agent include the amino acid sequence Tyr-Glu-Glu-Glu.

Particularly preferred hair treatment agents contemplated herein are characterized in that at least about 0.1 wt. %, especially at least about 0.5 wt. %, particularly preferably at least about 1 wt. %, more preferably at least about 2.5 wt. %, still more preferably at least about 5 wt. %, and in particular at least about 10 wt. % of all keratinic peptides present in the agent include the amino acid sequence Tyr-Glu-Glu-Glu-Ile.

Especially particularly preferred hair treatment agents contemplated herein are characterized in that at least about 0.1 wt. %, especially at least about 0.5 wt. %, particularly preferably at least about 1 wt. %, more preferably at least about 2.5 wt. %, still more preferably at least about 5 wt. %, and in particular at least about 10 wt. % of all keratinic peptides present in the agent include the amino acid sequence Tyr-Glu-Glu-Glu-Ile-Arg.

Still further preferred hair treatment agents contemplated herein are characterized in that at least about 0.1 wt. %, especially at least about 0.5 wt. %, particularly preferably at least about 1 wt. %, more preferably at least about 2.5 wt. %, still more preferably at least about 5 wt. %, and in particular at least about 10 wt. % of all keratinic peptides present in the agent include the amino acid sequence Tyr-Glu-Glu-Glu-Ile-Arg-Val.

In particular, preferred hair treatment agents contemplated herein are characterized in that at least about 0.1 wt. %, especially at least about 0.5 wt. %, particularly preferably at least about 1 wt. %, more preferably at least about 2.5 wt. %, still more preferably at least about 5 wt. %, and in particular at least about 10 wt. % of all keratinic peptides present in the agent include the amino acid sequence Tyr-Glu-Glu-Glu-Ile-Arg-Val-Leu.

The above-described conditions relate to the total content of peptides derived from keratinic materials in the agents contemplated herein. In addition to the oligopeptides of keratinic origin, it is possible, of course, to use further peptides and/or protein hydrolysates, for example from other native sources. For example, the additional use of wheat protein hydrolysates is preferred.

Suitable vitamins shall preferably be understood to mean the following vitamins, provitamins, and vitamin precursors, and the derivatives thereof:
Vitamin A: The group of substances referred to as vitamin A includes retinol (vitamin A₁) and 3,4-didehydroretinol (vitamin A₂). β-carotene is the provitamin of retinol. Possible vitamin A components are, for example, vitamin A acid and the esters thereof, vitamin A aldehyde, and vitamin A alcohol and the esters thereof, such as palmitate and acetate.
Vitamin B: The vitamin B group or vitamin B complex includes, among other things.
   Vitamin B₁ (thiamine)
   Vitamin B₂ (riboflavin)
   Vitamin B₃. The compounds nicotinic acid and nicotinic acid amide (niacinamide) are often covered by this designation.
   Vitamin B₅ (pantothenic acid and panthenol). Within this group, panthenol is preferably used. Derivatives of panthenol that can be used are in particular the esters and ethers of panthenol, pantolactone and cationically derivatized panthenols. Individual representatives are, for example, panthenol triacetate, panthenol monoethyl ether and the monoacetate thereof, and cationic panthenol derivatives.
   Vitamin B₆ (pyridoxine, pyridoxamine and pyridoxal).
Vitamin C (ascorbic acid): Use in the form of the palmitic acid ester, glucosides or phosphates can be preferred. Use in combination with tocopherols can likewise be preferred.
Vitamin E (tocopherols, in particular α-tocopherol).
Vitamin F: The term “vitamin F” is usually understood to mean essential fatty acids, in particular linoleic acid, linolenic acid and arachidonic acid.
Vitamin H: Vitamin H refers to the compound (3aS,4S,6aR)-2-oxohexahydrothienol[3,4-d]-imidazole-4-valeric acid, which, however, has since become established by the trivial name biotin.

Vitamins, provitamins and vitamin precursors from the groups A, B, E and H are particularly preferred. In particular, nicotinic acid amide, biotin, pantolactone and/or panthenol are preferred. The percentage by weight of the vitamin(s), vitamin derivative(s) and/or vitamin precursor(s) based the total weight of the hair treatment agents is preferably about 0.001 to about 2 wt. %, particularly preferably about 0.005 to about 1 wt. %, and in particular about 0.01 to about 0.5 wt. %.

Suitable plant extracts shall be understood to mean extracts that can be produced from all parts of a plant. These extracts are usually produced by extracting the entire plant. However, in isolated cases, it may also be preferred to produce the extracts exclusively from flowers and/or leaves of the plant.

Especially suited are the extracts of green tea, oak bark, stinging nettle, witch hazel, hops, chamomile, burdock root, horsetail, hawthorn, lime blossom, lychee, almond, aloe vera, pine needle, horse chestnut, sandalwood, juniper, coconut, mango, apricot, lime, wheat, kiwi, melon, orange, grapefruit, sage, rosemary, birch, mallow, lady's smock, creeping thyme, yarrow, thyme, balm, restharrow, coltsfoot, marshmallow, ginseng, ginger root, Echinacea purpurea, Olea europea, Boerhavia diffusa roots, Foeniculum vulgare and Apium graveolens.

Particularly preferred for use in the compositions contemplated herein are the extracts of green tea, stinging nettle, witch hazel, chamomile, aloe vera, ginseng, Echinacea purpurea, Olea europea and Boerhavia diffusa roots.

Extracting agents for producing the described plant extracts that may be used are water, alcohols and the mixtures thereof. Among the alcohols, lower alcohols, such as ethanol and isopropanol, in particular, however, polyhydric alcohols, such as ethylene glycol and propylene glycol, both as the sole extracting agent and also in a mixture with water, are preferred. Plant extracts based on water/propylene glycol at a ratio of about 1:10 to about 10:1 have proven to be particularly suitable.

The plant extracts can be used either in pure form or in dilute form. If they are used in dilute form, they usually contain approximately about 2 to about 80 wt. % active substance and, as solvent, the extracting agent or extracting agent mixture used during the extraction thereof.

The plant extracts may preferably be used in the hair treatment agents contemplated herein (based on the total weight of the agents) in an amount of about 0.01 to about 10 wt. %, more preferably of about 0.05 to about 7.5 wt. %, and in particular of about 0.1 to about 5 wt. %.

Glycerol can be added separately to the hair cleaning and care agents in an amount of up to about 10 wt. % (based on the total weight of the agent). However, it can also be a component of the aforementioned aqueous-alcoholic carrier.

It was found that the hair treatments agents as contemplated herein are also suitable for use as an anti-dandruff preparation. The total weight of anti-dandruff agents based on the total weight of the hair treatment agents can preferably be about 0.01 to about 10 wt. %, more preferably about 0.025 to about 7.5 wt. %, particularly preferably about 0.05 to about 5 wt. %, and in particular about 0.075 to about 3 wt. %. Suitable active anti-dandruff ingredients can be selected from piroctone olamine, climbazole, zinc pyrithione, ketoconazoles, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts and/or arnica extracts.

Climbazole, zinc pyrithione and piroctone olamine are preferred.

Further active ingredients, auxiliary substances and additives that can preferably be present in the hair treatment agents contemplated herein are, for example:
humectants;
perfumes; UV filters;
thickening agents such as gelatin or gums, for example agar-agar, guar gum, alginates, xanthan gum, gum arabic, karaya gum, locust bean gum, linseed gums, dextrans, cellulose derivatives, for example methyl cellulose, hydroxyalkyl cellulose and carboxymethyl cellulose, starch fractions and derivatives, such as amylose, amylopectin and dextrins, clays and phyllosilicates, such as bentonite or fully synthetic hydrocolloids, such as polyvinyl alcohol, the Ca, Mg or Zn soaps;
structurants such as maleic acid and lactic acid;
dimethyl isosorbide;
cyclodextrins;
fiber structure-improving active ingredients, in particular monosaccharides, disaccharides and oligosaccharides such as glucose, galactose, fructose, fructose and lactose; dyes for coloring the agent;
active ingredients such as bisabolol and/or allantoin;
complexing agents such as EDTA, NTA, 1-alanine diacetic acid, and phosphonic acids; ceramides. Ceramides shall be understood to mean N-acylsphingosine (fatty acid amides of sphingosine) or synthetic analogs of such lipids (known as pseudoceramides); propellants such as propane-butane mixtures, N₂O, dimethylether, CO₂ and air;
antioxidants;
additional viscosity regulators such as salts (NaCl).

The agents contemplated herein are preferably so-called rinse-off products, which is to say they are rinsed off the hair again after a certain exposure time. This exposure time is preferably less than one hour, which is to say the user preferably does not leave the products on the hair until the next time the hair is washed.

A further embodiment of the present invention is thus a method for treating hair, in which an agent as contemplated herein is applied to dry or moist hair, where it is left for a period of about 30 to about 300 seconds and then rinsed off.

The agents as contemplated herein result in considerable greater stability of artificial colorings against washout of the dye. In this way, chemically colored hair may be washed considerably more frequently with the agents contemplated herein, without resulting in undesirable bleeding or fading of the due.

A further embodiment of the present invention is thus the use of agents as contemplated herein to reduce dye from being washed out of chemically colored hair.

What was said above with respect to the agents contemplated herein applies, mutatis mutandis, with respect to preferred embodiments of the method contemplated herein and the use contemplated herein.

### Examples

All information in wt. %

Hair shampoos series 1

| | 1 | 2 | 3 | 4 | 5 | 6 |
|---|---|---|---|---|---|---|
| Sodium laureth sulfate (AS) | 10.0 | 10.0 | 10.0 | 10.0 | 10.0 | 10.0 |
| Cocamidopropyl betaine (AS) | 4.0 | 4.0 | 4.0 | 4.0 | 4.0 | 4.0 |
| Ammonium lauryl sulfate | 4.0 | 4.0 | 4.0 | 4.0 | 4.0 | 4.0 |
| Polyquaternium-10 | 0.3 | 0.3 | 0.3 | 0.3 | 0.3 | 0.3 |
| Cocamide MEA (AS) | 0.8 | 0.8 | 0.8 | 0.8 | 0.8 | 0.8 |
| PEG-7 glyceryl cocoate | 1.0 | 1.0 | 1.0 | 1.0 | 1.0 | 1.0 |
| Nicotinic acid amide | 0.3 | 0.3 | 0.3 | 0.3 | 0.3 | 0.3 |
| Citric acid | 0.5 | 0.5 | 0.5 | 0.5 | 0.5 | 0.5 |
| Calcium sulfate | 0.5 | 0.5 | 0.5 | 0.5 | 0.5 | 0.5 |
| Panthenol | 0.5 | 0.5 | 0.5 | 0.5 | 0.5 | 0.5 |
| Laureth-2 | 1.2 | 1.2 | 1.2 | 1.2 | 1.2 | 1.2 |
| Sodium chloride | 1.3 | 1.3 | 1.3 | 1.3 | 1.3 | 1.3 |
| Ethyl lauroyl arginate | 0.3 | 0.3 | 0.3 | 0.3 | 0.3 | 0.3 |
| Dimethyloxazolidine | — | 0.05 | 0.05 | 0.05 | 0.05 | 0.05 |
| Glutaraldehyde | — | 0.05 | 0.06 | 0.07 | 0.08 | 0.1 |
| Hexetidine | — | 0.05 | 0.06 | 0.07 | 0.08 | 0.1 |
| Phenoxyethanol | — | 0.05 | 0.05 | 0.05 | 0.05 | 0.05 |
| Phenoxyisopropanol | — | 1.0 | 0.5 | 0.25 | 0.2 | 0.1 |
| o-cymen-5-ol | — | 0.1 | 0.05 | 0.1 | 0.05 | 0.1 |
| o-phenylphenol | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | to make 100 |

Hair shampoos series 2

| | 1 | 2 | 3 | 4 | 5 | 6 |
|---|---|---|---|---|---|---|
| Sodium laureth sulfate (AS) | 7.0 | 7.0 | 7.0 | 7.0 | 7.0 | 7.0 |
| Disodium cocoamphodiacetate | 4.0 | 4.0 | 4.0 | 4.0 | 4.0 | 4.0 |
| (AS) | | | | | | |
| Cocamide MEA (AS) | 0.3 | 0.3 | 0.3 | 0.3 | 0.3 | 0.3 |
| Guar hydroxypropyl trimonium | 0.1 | 0.1 | 0.1 | 0.1 | 0.1 | 0.1 |
| chloride | | | | | | |
| Panthenol | 0.2 | 0.2 | 0.2 | 0.2 | 0.2 | 0.2 |
| Magnesium chloride | 0.8 | 0.8 | 0.8 | 0.8 | 0.8 | 0.8 |
| Whey protein hydrolysate | 0.3 | 0.3 | 0.3 | 0.3 | 0.3 | 0.3 |
| Lactic acid | 0.8 | 0.8 | 0.8 | 0.8 | 0.8 | 0.8 |
| PEG-120 methyl glucose | 0.7 | 0.7 | 0.7 | 0.7 | 0.7 | 0.7 |
| dioleate | | | | | | |
| Ethyl lauroyl arginate | 0.4 | 0.4 | 0.4 | 0.4 | 0.4 | 0.4 |
| Dimethyloxazolidine | — | 0.4 | 0.3 | 0.2 | 0.1 | 0.05 |
| Glutaraldehyde | — | 0.05 | 0.06 | 0.07 | 0.08 | 0.1 |
| Hexetidine | — | 0.05 | 0.06 | 0.07 | 0.08 | 0.1 |
| Phenoxyethanol | — | 0.05 | 0.05 | 0.05 | 0.05 | 0.05 |
| Phenoxyisopropanol | — | 1.0 | 0.5 | 0.25 | 0.2 | 0.1 |
| o-cymen-5-ol | — | 0.1 | 0.05 | 0.1 | 0.05 | 0.1 |
| o-phenylphenol | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | to make 100 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A hair treatment agent, comprising:
at least one anionic surfactant;
at least one amphoteric surfactant;
at least one divalent or trivalent metal salt;
at least one organic acid; and
ethyl lauroyl arginate.

2. The hair treatment agent according to claim 1, comprising, based on the weight thereof, about 0.5 to about 20 wt. % of the at least one anionic surfactant(s).

3. The hair treatment agent according to claim 1, comprising, based on the weight thereof, about 0.5 to about 20 wt. % alkyl (ether) sulfates of the general formula R—(OCH₂—CH₂)ₙ—OSO₃X, in which R denotes a straight-chain or branched, saturated or unsaturated alkyl group having 8 to 24 carbon atoms, n denotes the number 0 or 1 to 12, and X denotes an alkali, alkaline earth, ammonium or alkanolamine ion.

4. The hair treatment agent according to claim 1, by comprising, based on the weight thereof, about 0.3 to about 10 wt. % of the at least one amphoteric surfactant(s).

5. The hair treatment agent according to claim 1, by comprising, based on the weight thereof, about 0.01 to about 10 wt. % of at least one divalent or trivalent metal salt from the group of the organic or inorganic copper, zinc, iron(II), calcium, magnesium, iron(III) and/or aluminum salts.

6. The hair treatment agent according to claim 1, comprising, based on the weight thereof, about 0.001 to about 10 wt. % of the at least one organic acid(s) from the group consisting of formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluoyl acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrole carboxylic acid, 1,2,4,6,7-napthalene pentaacetic acid, malonaldehyde acid, 4-hydroxyphthalimide acid, 1-pyrazole carboxylic acid, gallic acid or propanetricarboxylic acid, glycolic acid, lactic acid, malic acid, tartaric acid or citric acid.

7. The hair treatment agent according to claim 6, comprising, based on the weight thereof, about 0.001 to about 10 wt. % of the at least one organic acid(s) from the group consisting of formic acid, oxalic acid, maleic acid, lactic acid, tartaric acid or citric acid.

8. The hair treatment agent according to claim 1, comprising, based on the weight thereof, about 0.001 to about 2 wt. % ethyl lauroyl arginate HCl.

9. A method for treating hair, the method comprising the steps of:
applying to dry or moist hair a hair treatment agent comprising:
an anionic surfactant;
an amphoteric surfactant;
a divalent or trivalent metal salt;
an organic acid; and
ethyl lauroyl arginate, and
rinsing the hair treatment agent from the hair after the hair treatment agent is left on the hair for a period of from about 30 to about 300 seconds.

10. (canceled)

11. The hair treatment agent according to claim 2, comprising, based on the weight thereof, about 0.75 to about 15 wt. % of the at least one anionic surfactant(s).

12. The hair treatment agent according to claim 11, comprising, based on the weight thereof, about 1 to about 12 wt. % of the at least one anionic surfactant(s).

13. The hair treatment agent according to claim 12, comprising, based on the weight thereof, about 2 to about 10 wt. % the at least one anionic surfactant.

14. The hair treatment agent according to claim 3, comprising, based on the weight thereof, about 2 to about 10 wt. % alkyl (ether) sulfates of the general formula R—(OCH₂—CH₂)ₙ—OSO₃X, in which R denotes a straight-chain or branched, saturated or unsaturated alkyl group having 8 to 24 carbon atoms, n denotes the number 0 or 1 to 12, and X denotes an alkali, alkaline earth, ammonium or alkanolamine ion.

15. The hair treatment agent according to claim 4, comprising, based on the weight thereof, about 1 to about 5 wt. % of the at least one amphoteric surfactant(s).

16. The hair treatment agent according to claim 5, comprising, based on the weight thereof, about 0.3 to about 3 wt. % of at least one divalent or trivalent metal salt from the group of the organic or inorganic copper, zinc, iron(II), calcium, magnesium, iron(III) and/or aluminum salts.

17. The hair treatment agent according to claim 6, comprising, based on the weight thereof, about 0.1 to about 2 wt. % of the at least one organic acid(s) from the group consisting of formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluoyl acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrole carboxylic acid, 1,2,4,6,7-napthalene pentaacetic acid, malonaldehyde acid, 4-hydroxyphthalimide acid, 1-pyrazole carboxylic acid, gallic acid or propanetricarboxylic acid, glycolic acid, lactic acid, malic acid, tartaric acid or citric acid.

18. The hair treatment agent according to claim 7, comprising, based on the weight thereof, about 0.1 to about 2 wt. % of the at least one organic acid(s) from the group consisting of formic acid, oxalic acid, maleic acid, lactic acid, tartaric acid or citric acid.

19. The hair treatment agent according to claim 8, comprising, based on the weight thereof, about 0.01 to about 1 wt. % ethyl lauroyl arginate HCl.

20. The hair treatment agent according to claim 19, comprising, based on the weight thereof, about 0.025 to about 0.6 wt. % ethyl lauroyl arginate HCl.

21. The hair treatment agent according to claim 20, comprising, based on the weight thereof, about 0.05 to about 0.4 wt. % ethyl lauroyl arginate HCl.
